(19) United States
(12) Patent Application Publication
(10) Pub. No.: US 2019/0015591 A1
(43) Pub. Date: Jan. 17, 2019

(54) INJECTION DEVICE

(71) Applicant: H&B ELECTRONIC GMBH & CO. KG, Deckenpfronn (DE)

(72) Inventors: Tobias MORLOK, Mötzingen (DE); Wilfried WEBER, Schopfloch (DE); Dariusz PETRY, Aidlingen (DE); Wolfgang STAUSS, Rangendingen (DE)

(73) Assignee: H&B ELECTRONIC GMBH & CO. KG, Deckenpfronn (DE)

(21) Appl. No.: 16/064,217

(22) PCT Filed: Dec. 14, 2016
(86) PCT No.: PCT/EP2016/080932

§ 371 Date: Jun. 20, 2018

(30) Foreign Application Priority Data
Feb. 3, 2016 (DE) 20 2016 100 531.0

(51) Int. Cl. A61M 5/20; A61M 5/24; A61M 5/32; A61M 5/315

(52) U.S. Cl. CPC A61M 5/20; A61M 5/24; A61M 5/326; A61M 2005/3152; A61M 5/31511; A61M 2005/206; A61M 2005/2414; A61M 5/3287

(57) ABSTRACT

An injection device has a housing, in which a syringe can be held, which has an injection needle, a holding chamber, a plunger tip, and a syringe plunger; and a control assembly for the controlled actuation of the syringe during an application process, which includes at least an insertion stroke, an injection stroke, and a return stroke. The control assembly has an actuation element for receiving a manual driving force, a syringe holder, which can be slid in relation to the housing and to which the holding chamber can be fastened, a plunger holder, which can be slid in relation to the housing and to which the syringe plunger can be fastened, and a transmission device, which has a gearing for coupling the plunger holder to the actuation element, by means of which gearing the plunger holder can be driven in accordance with a motion of the actuation element relative to the housing. The housing has a first outer housing part and a second outer housing part, which are retained on each other in such a way that said housing parts can be moved between an open position, in which the syringe holder is accessible, and a closed position, in which the syringe holder is enclosed.

The invention relates to an injection device according to the preamble of claim 1. It has a housing in which a syringe having an injection needle, a barrel, a piston and a syringe plunger can be received. The injection device further comprises a control assembly for the controlled actuation of the syringe during an application procedure comprising at least an insertion stroke, an injection stroke and a return stroke. The control assembly has an actuation element for acting, through a manual driving force, a syringe holder that can be displaced relative to the housing and to which the barrel can be fastened, as well as a plunger holder that can be displaced relative to the housing and to which the syringe plunger can be attached. Furthermore, a transmission device is provided on the injection device, which has a gear drive for connecting the plunger holder to the actuation element. By means of the gear drive, the plunger holder can be driven in accordance with relative motion of the actuation element.

An injection device for the simplified administration of a medicament from a syringe is known from DE 202014004561 U1. The filled syringe is housed inaccessibly within the injection device. Further, an actuation element is provided that can be pressed by manual force. A gear drive is arranged between this actuation element and a housing of the injection device, by means of which a plunger holder can be driven to push out the syringe, in accordance with relative movement of the actuation element with respect to the housing.

Injection devices of this kind are sold together with the medicament they contain, so that the injection device must be made available by the medicament manufacturer or filler. This means that the injection device also has to be stored and transported together with the medicament. Depending on the type of medicament used, it may also be necessary for it, together with the injection device, to be refrigerated during storage or transport. Due to the fixed connection of the syringe in the injection device, the refrigeration space thus required is determined not just by the syringes and the medicament they contain, but also by the much bigger injection device. As a result, the refrigeration space required for medicaments administered by such injection devices is relatively large. Moreover, there is a need in the market for injection devices to be capable of being marketed independently of any particular medicament.

DE 203 11 996 U1 discloses an injection device in which a syringe with the medicament to be administered can be used to make controlled insertion, injection and return strokes by simple, directed application of force. The drive force is applied by manually actuating an actuation element, by means of which a syringe holder and a plunger holder are displaced.

Further injection devices with automatic drive are known from DE 202010000846 U1, DE 69839165 T1 and U.S. Pat. No. 9,180,258 B2, which devices have a two-part external housing. The outer housing can be displaced between open and closed positions, which prevents actuation of the drive when the outer housing is in the open position.

The aim of the invention is to avoid the above-mentioned disadvantages of an injection device of this kind and to enable its medicament-independent marketing as well as to simplify the storage and transport of the medicament administered in it or to reduce the costs arising.

This aim is achieved by an injection device with the features of claim 1. The housing has a first outer housing part and a second outer housing part that are retained on each other. The two outer housing parts can be displaced with respect to each other between an open position, in which the syringe holder is accessible, and a closed position, in which the syringe holder is enclosed.

Here, a pivot bearing is provided between the first outer housing part and the second outer housing part. This facilitates displacement of the two housing parts between the open position and the closed position by particularly simple and cost-effective means. In addition, in this way the housing parts can be manufactured more cost-effectively, for example by a single moulding operation and easier fitting of the other components in the open position is enabled.

Here, it is advantageous if the pivot bearing is provided with a film hinge or is formed by such a hinge. Through such a film hinge, for example in the form of a butterfly hinge, both the pivot bearing and double biasing of it can be achieved particularly cost-effectively. Alternatively, the pivot bearing can be provided with lockable hinge means. This enables separate manufacture and simple assembly of the two outer housings.

In this way, it is possible to market, store and transport the injection device independently of the syringe to be housed in it or the medicament it contains. The injection device can then be available in a pharmacy, irrespective of the medicament concerned. This means that, if required, a doctor could prescribe to the patient the injection device separately or the patient can buy the injection device at his own expense in order to be able to inject the medicament more comfortably. In the case of a medicament that needs to be refrigerated, this can be stored and transported in a refrigerated condition, separately from the injection device. By contrast, the injection device itself can be stored and transported without refrigeration. The required refrigeration space is thus determined just by the medicament or the external dimensions of the syringes in which the medicament is held. Before the medicament is administered, an appropriate syringe can be inserted into the injection device that has been brought into the open position. The second outer housing part is then moved into the closed position relative to the first outer housing part, in which position the injection device is then ready for use. To that end, the transmission device between the plunger holder and the actuation element is also provided with a coupling device comprising coupling means on the plunger holder side and a counter-coupling means on the actuation element side, and that is disconnected in the open position. In this way, accidental application of force to the syringe can be prevented when the outer housing parts are in the open position.

In a particularly advantageous embodiment, both of the outer housing parts are biased towards each other, both in the open position and in the closed position. Through such bistable positioning of the two outer housing parts, in the open position the syringe can be inserted particularly easily, while in the closed position the syringe is safely housed and protected in the interior of the injection device.

It is advantageous if the second outer housing part can be permanently or releasably locked to the first outer housing part in the closed position. In this way, accidental opening of the housing during use can be avoided. In this case, the second outer housing part is, for example, permanently connected to the first outer housing part in the closed position. This ensures use of the injection device as a single-use product. To that end, a permanent snap coupling between the first outer housing part and the second outer housing part is preferably provided, which effectively prevents opening of the injection device after the medicament has been administered by the injection device configured as a single-use product. In an alternative embodiment of the injection device, the first outer housing part and the second outer housing part can be locked by a sliding finger support that is permanently connected to the housing. In this case, the separate finger support enables particularly simple manufacture of the means required to achieve the permanent lock. In another alternative embodiment of the injection device, the second outer housing part can be releasably connected to the first outer housing part in the closed position. The releasable connection between the two housing parts enables repeated use of the injection device. Preferably, in this case a releasable snap coupling is provided between the first outer housing part and the second outer housing part, which ensures automatic and stable connection between the two housing parts in the closed position. Alternatively, the first outer housing part and the second outer housing part can be locked in the closed position by a finger support that can be releasably slid onto the housing. Such a finger support enables particularly simple and stable releasable connection of the two outer housing parts. For both permanent and releasable locking of the two housing parts by the finger support, the finger support can be connected to or guided on the rest of the injection device so that it cannot be lost from the injection device both before and/or after locking.

Preferably, an end-stop is formed between the first outer housing part and the second outer housing part, by means of which the two outer housing parts can be positioned next to each other in the open position. By means of this end-stop, when the two outer housing parts are moved into the open position overloading of the pivot bearing, in particular overstretching of the material, can be prevented.

In another advantageous embodiment, the first outer housing part and the second outer housing part are guided next to each other when pivoting from the open position to the closed position, whereby a stable pivot movement to the precise closed position is ensured. In that case, it is also advantageous if strain relief means that work transverse to the pivoting direction are provided between the first outer housing part and the second outer housing part. Such strain relief means can, for instance, be formed by elements that can be brought into engagement with each other, by means of which an effective closure can be achieved along the principal extent of the injection device. In this way, the pivot bearing located between the two outer housings can be protected by the stress relief means against damage caused in use by the application of an external force. It is also advantageous if the actuation element is supported in the closed position on the plunger holder. The actuation element can thereby be stabilised by the plunger holder, in particular to exhibit sufficient torsional and flexural rigidity with respect to the forces applied during use. It is also advantageous if the plunger holder has a contact portion to lie on the syringe tip, on which contact portion lateral support means are formed. Lateral support means of this kind can be used to centre the syringe tip in the plunger holder, to ensure exact application of force to it through the plunger holder during use.

It is also advantageous if the pivot bearing constitutes a defined breaking point. In this way, it is possible to design the injection device for a prescribed number of uses and to show that this predefined number has been reached by breaking of the pivot bearing. As a result, it is possible to bring to an end the useability of the injection device, for instance before flaws in the control assembly can occur.

Moreover, it is advantageous if an actuation lock is provided that in the open position prevents movement of the actuation element. This can prevent both misuses of the injection device as well as injuries to the operator. Preferably, the actuation lock has a locking mechanism that is unlocked in the closed position. With such a locking mechanism, the actuation lock can be automatically unlocked simply solely by moving the outer housing parts to the closed position respectively by attaching the finger rest.

Further, the syringe holder and the plunger holder are held on the first outer housing part and the actuation element and the gear drive are held on the second outer housing part.

Here, it is beneficial if the coupling means on the plunger holder side and the counter-coupling means on the actuation element side, can in the closed position be brought into positive engagement that acts in and counter to the direction of the injection. Through a coupling device formed in this way, a releasable yet stable transfer of force from the actuation element to the plunger holder can be ensured.

In a particularly preferred embodiment of the injection device, adapter means are provided, by means of which the syringe holder and/or the plunger holder can be adapted to different syringes. In particular, with such adapter means it is possible to adapt the injection device to syringes with different lengths. Alternatively or in addition to this, the syringe holder and/or the plunger holder can be exchangeable. In this way, syringe holders or plunger holders of the appropriate size can be used for different syringes.

In another advantageous embodiment of the injection device, encoding means are provided between the outer housing parts and the syringe holder and/or the plunger holder, which define a starting position in which the outer housing parts can be brought into the closed position. This can ensure that the outer housing parts can be moved to the closed position only when the syringe holder and/or the plunger holder are in the said starting position.

Furthermore, a locking mechanism is provided between the first outer housing part and the second outer housing part, which can prevent the two outer housing parts being moved into the open position while the injection device is being operated. By this means, safe use of the injection device can be ensured. Preferably, the locking mechanism has a rear-engaging element on one of the outer housing parts, by means of which an edge of a securing groove of the other housing part can be gripped. It is beneficial if the securing groove is inset on the plunger holder and the rear-engaging element forms encoding means on the actuation element side, the securing groove having an entry opening that, in the starting position of the syringe holder, is located level with the encoding means. This can effectively prevent opening of the outer housing parts during use of the injection device.

Preferably, a releasable connecting mechanism is located between the syringe holder and the plunger holder, which has a latch element that can be brought into engagement with a corresponding latch holder in an insertion position and in a return position. In this way, movement of the syringe holder can be securely coupled to the plunger holder during an insertion stroke as well as during a return stroke. It is preferable if the latch element is formed as an engaging hook held by an elastic arm on the plunger holder, which hook is automatically controlled by a control profile formed on the syringe holder. In this way, the coupling and decoupling of the plunger holder with the syringe holder can be controlled precisely in the course of the various strokes during use. Preferably, the elastic arm is held without tension in the insertion position. As a result, the injection device can be kept ready for operation respectively stored over a longer period of time without the material of the elastic arm becoming fatigued.

In another advantageous embodiment of the injection device, a displacement mechanism is provided, by means of which the plunger holder can be automatically brought to the starting position when the housing parts are moved into the open position. With such a displacement mechanism, an injection device designed for repeated use can be brought into the starting position simply by opening the outer housing parts so that it can be made ready for use again after the used syringe has been replaced by a new one and displacement of the outer housing parts to the closed position. Preferably, the displacement mechanism has a spring device that in the open position biases the plunger holder to the starting position. At the same time, the coupling device on the plunger holder side and the actuation element with the counter-coupling device on the actuation element side are moved into the starting position. As a result, the injection device can be prepared for another use very easily. It is beneficial for fixing means to be provided between the housing and the plunger holder, by means of which in the starting position at least the plunger holder can be fixed relative to the housing. In this way, the injection device can be reliably held in the starting position.

In another advantageous embodiment of the injection device, the actuation element is coupled to a damping device. By this means, the displacement speed of the actuation element during use can be appropriately limited. In such a case, the damping device has a linearly acting air damper, by means of which a displacer, the movement of which is coupled to the actuation element, can be displaced along an airspace. By this means, the damping device can be manufactured particularly cost-effectively.

It is also beneficial for an end-position stop to be provided, by means of which the actuation element can be held in an end position taken up upon completion of an injection process. By this means, accidental further movement of the actuation element after completion of an injection procedure can be prevented. In this case, the end-position lock can be brought into a release position by moving the outer housing parts into the open position. In this way, the actuation element can be moved back to its starting position after the housing has been opened to bring the injection device into a ready-to-use condition.

The Figures show an exemplary embodiment of the invention. They show:

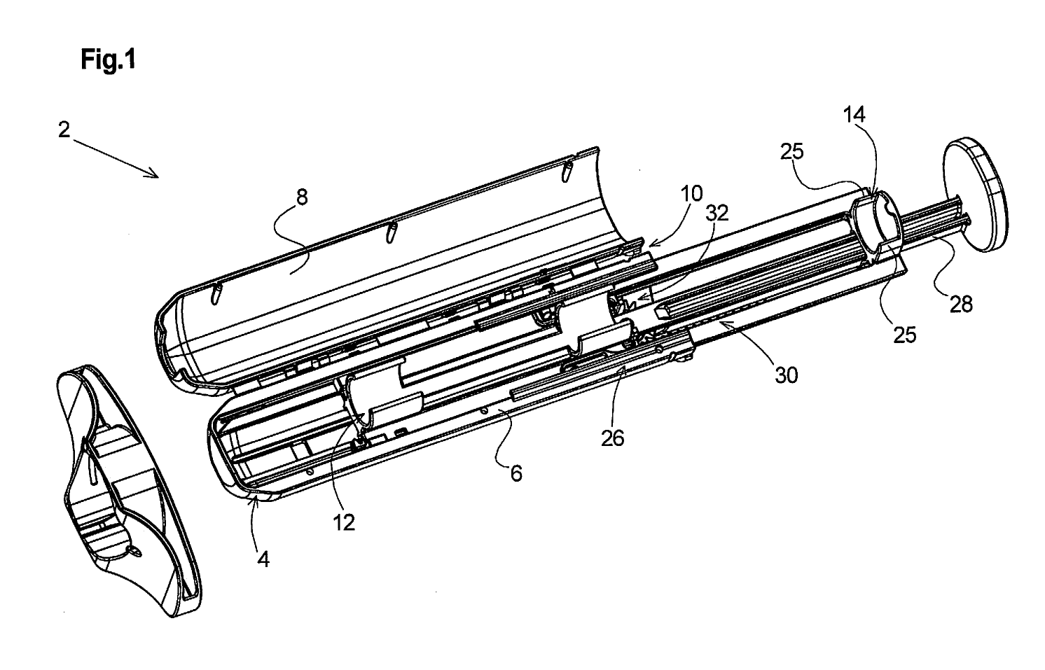

FIG. 1 a perspective view of an injection device according to the invention in an open position,

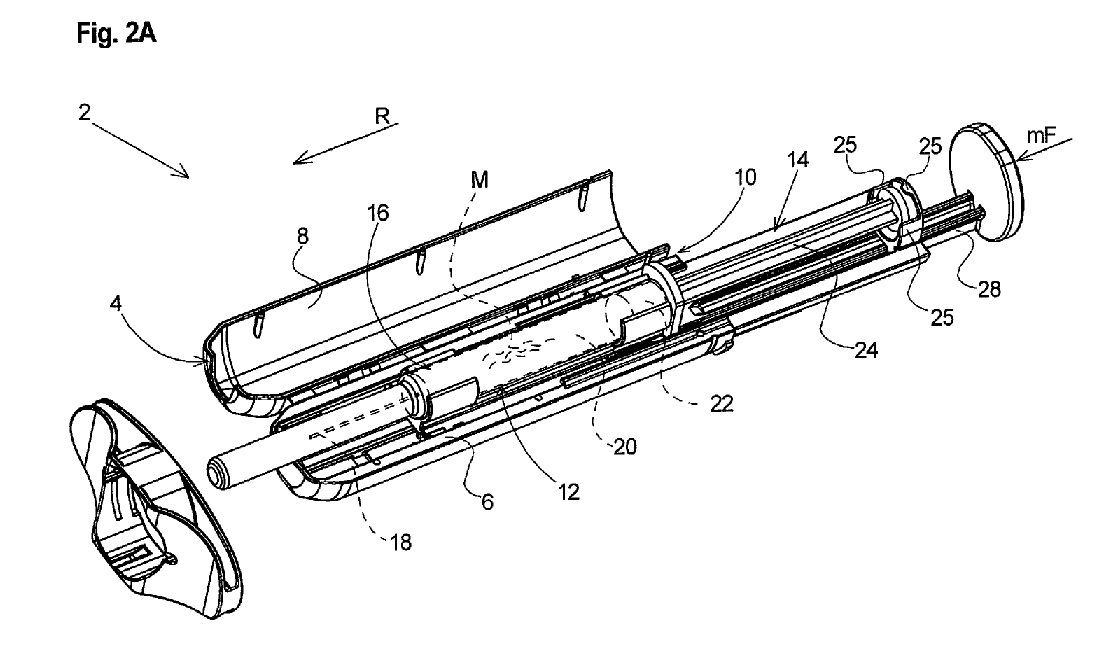

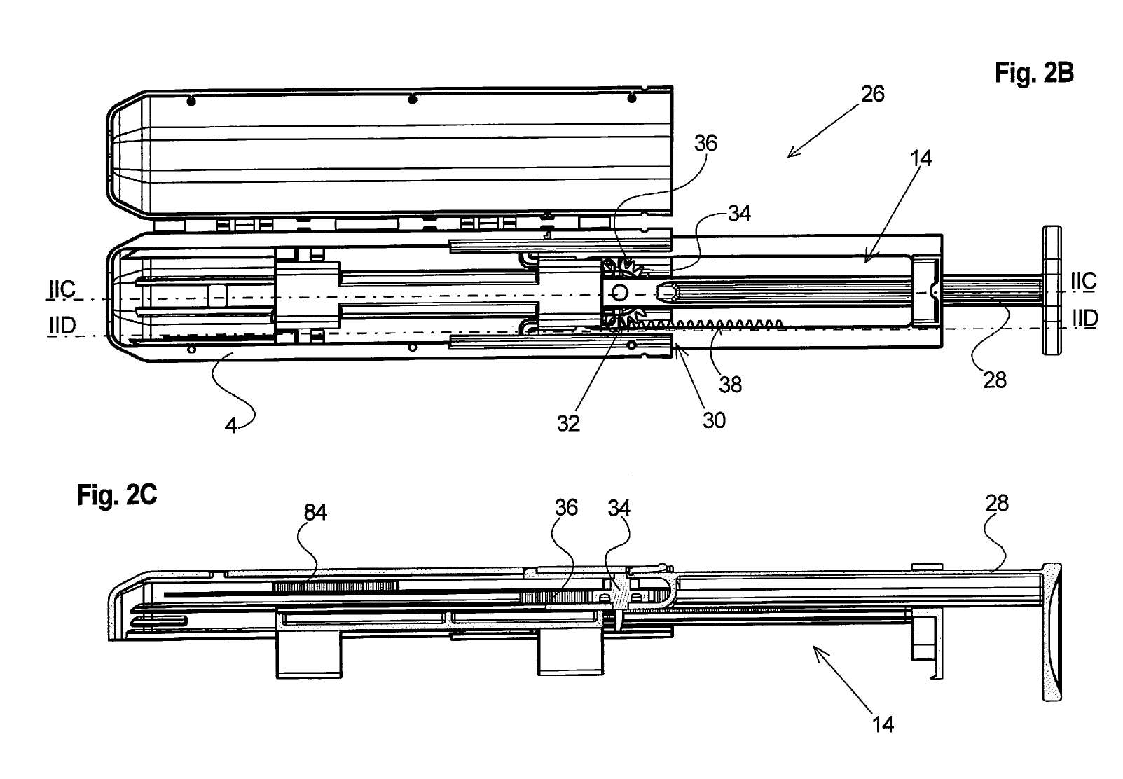

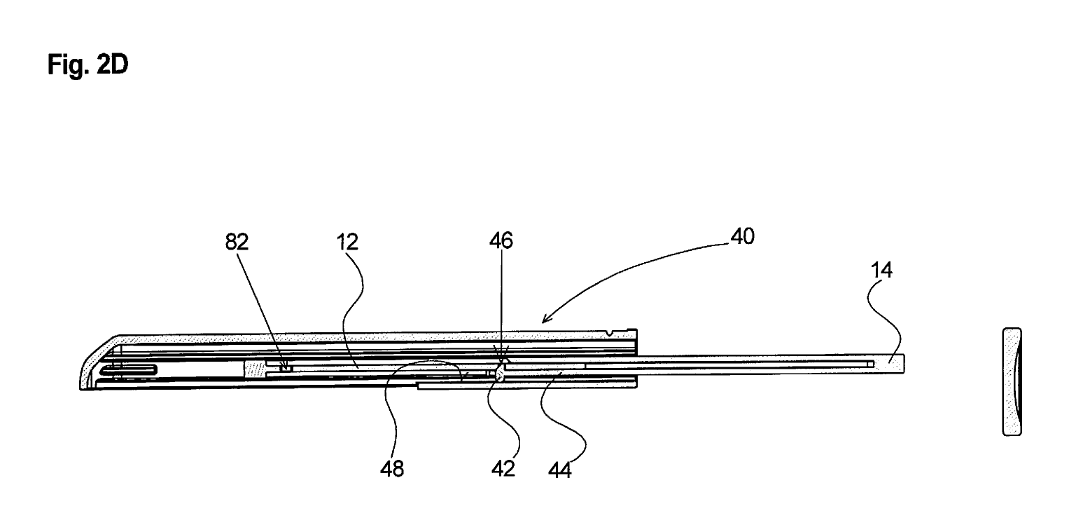

FIG. 2A a perspective view of the injection device according to FIG. 1 with a syringe inserted,

FIG. 2B a plan view of the injection device according to FIG. 1,

FIG. 2C a cut-away view of the injection device in the plane IIC-IIC of FIG. 2B,

FIG. 2D a cut-away view of the injection device in the plane IID-IID of FIG. 2B,

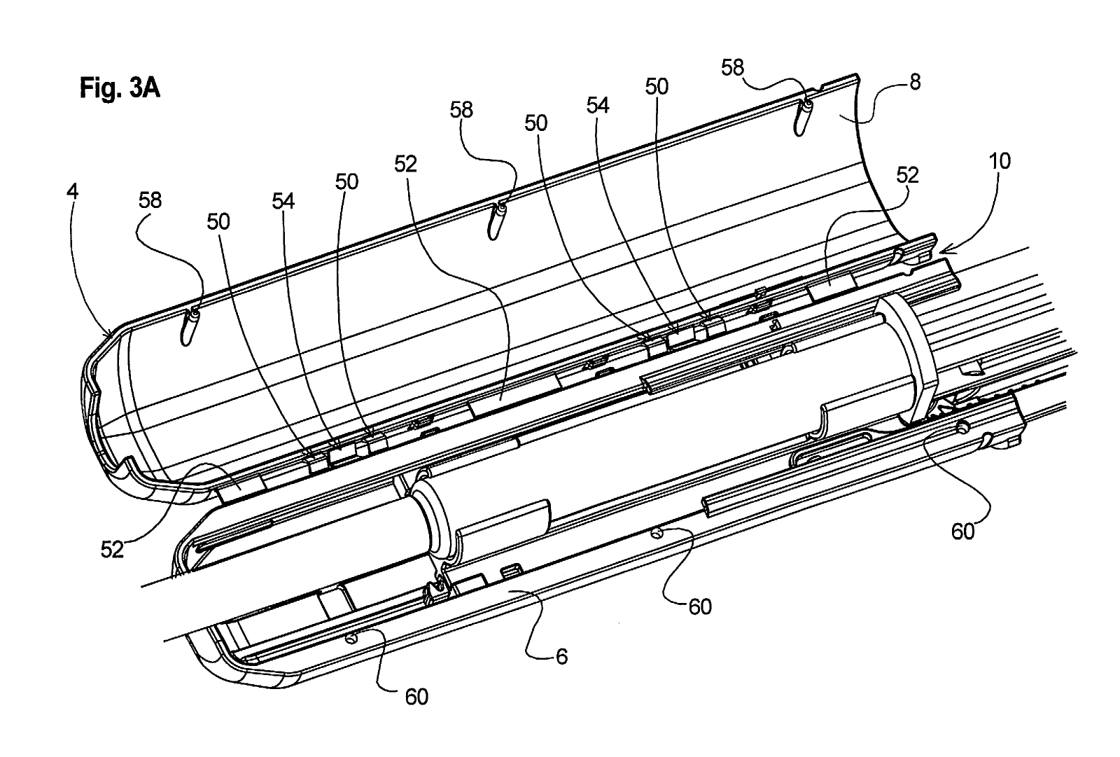

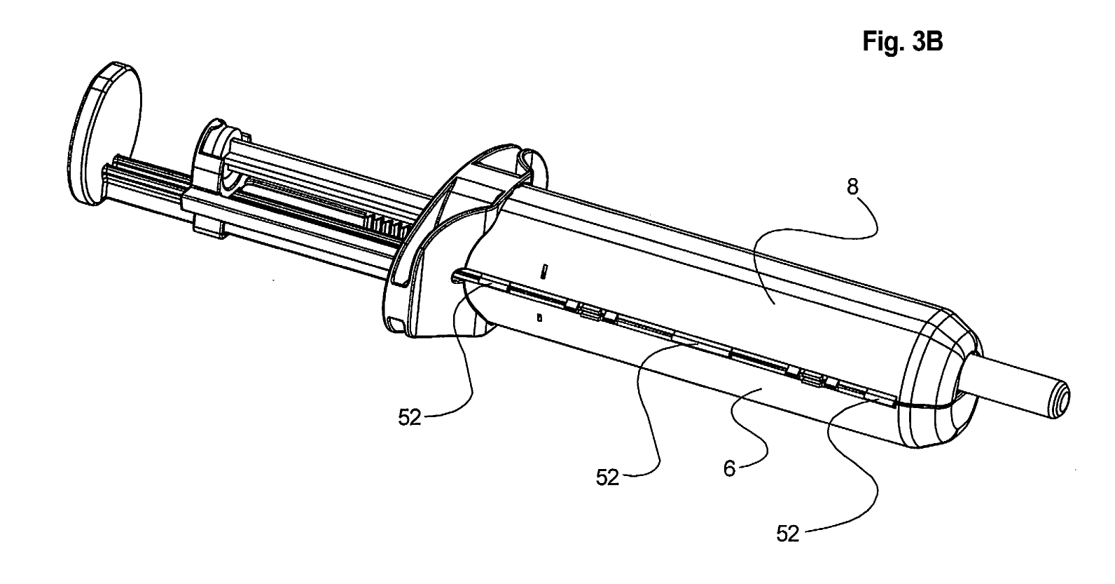

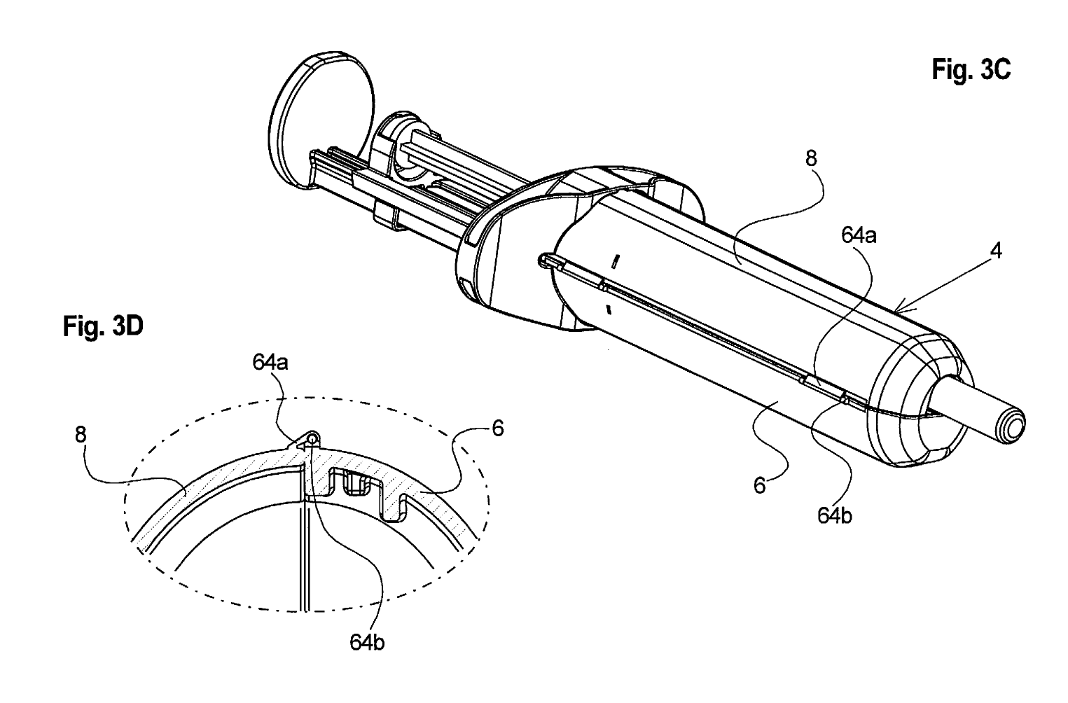

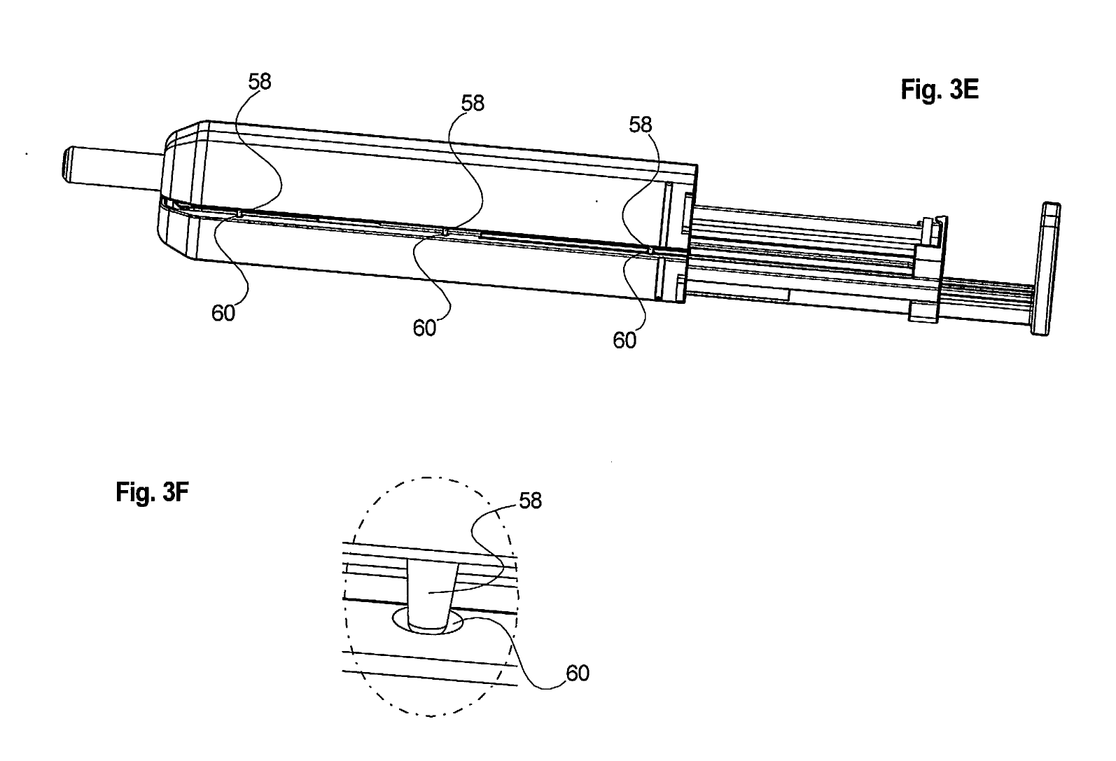

FIG. 3A a perspective view of a housing of the injection device according to FIG. 1 in the open position,

FIG. 3B a perspective view of the injection device according to FIG. 1 in a closed position,

FIG. 3C a perspective view of the injection device according to FIG. 3B with an alternative embodiment of a pivot bearing,

FIG. 3D an enlarged cut-away view through the pivot bearing according to FIG. 3C,

FIG. 3E a side view of the injection device according to FIG. 1 when being brought into the closed position,

FIG. 3F an enlarged view of guide means of the injection device according to FIG. 3 when being brought into the closed position,

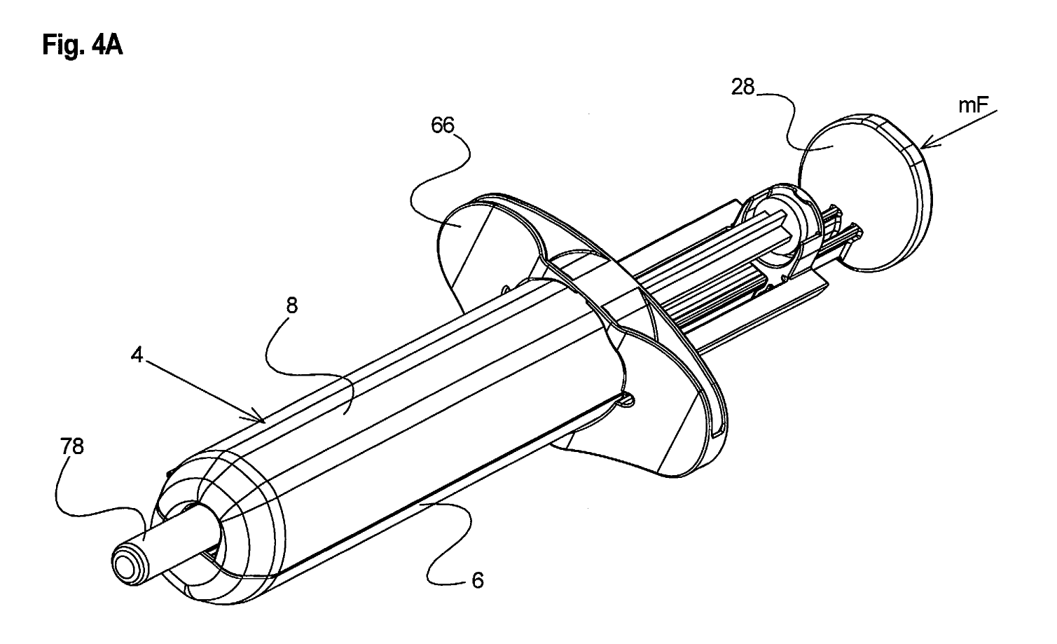

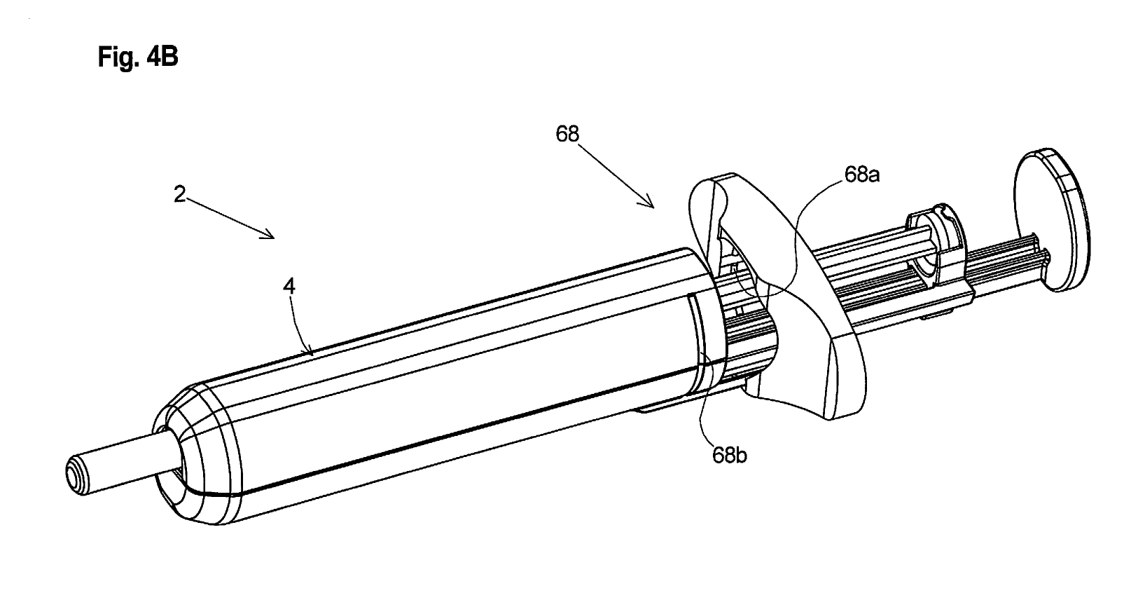

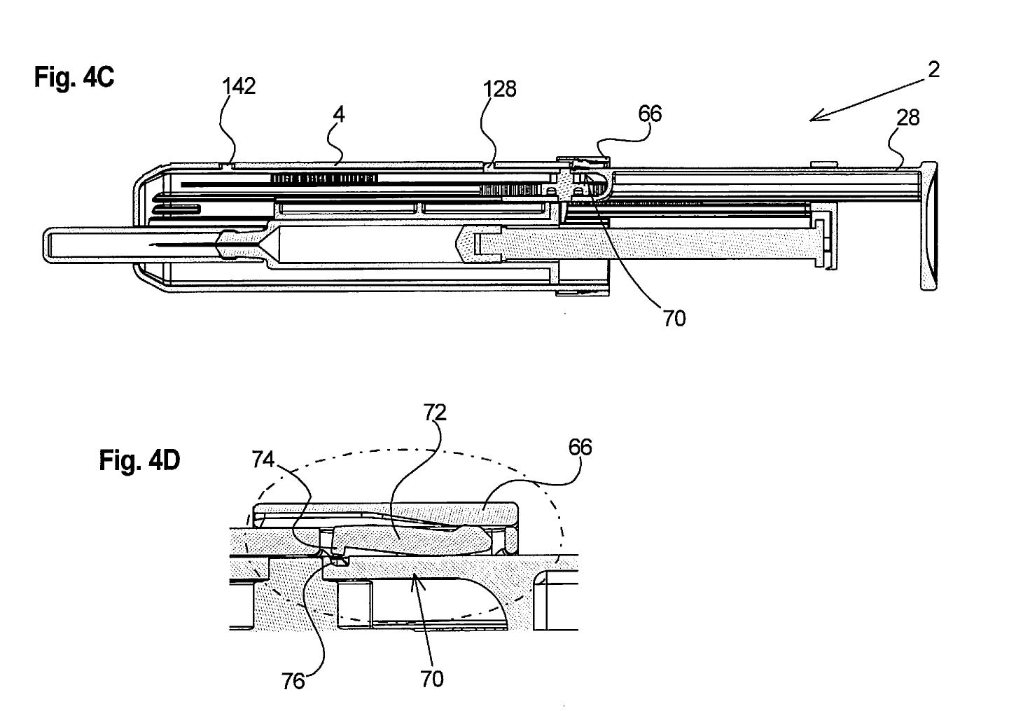

FIG. 4A a perspective view of the injection device according to FIG. 1 in a starting position,

FIG. 4B a perspective view of the injection device according to FIG. 1 with loose finger support,

FIG. 4C a longitudinal section through the injection device according to FIG. 4A,

FIG. 4D an enlarged view of an actuation lock of the injection device of FIG. 4C,

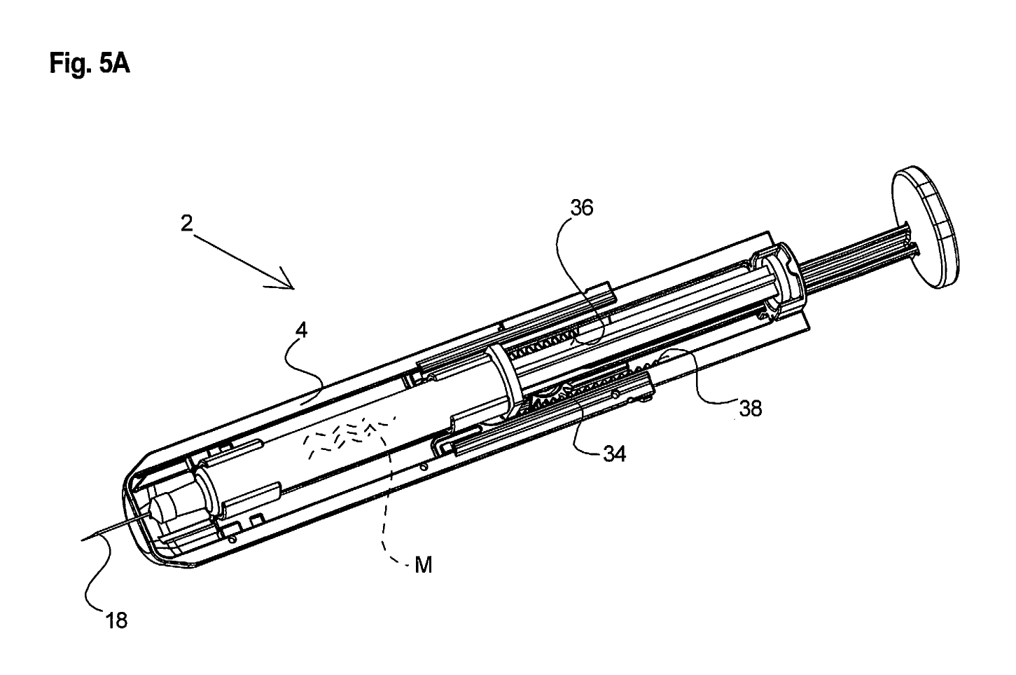

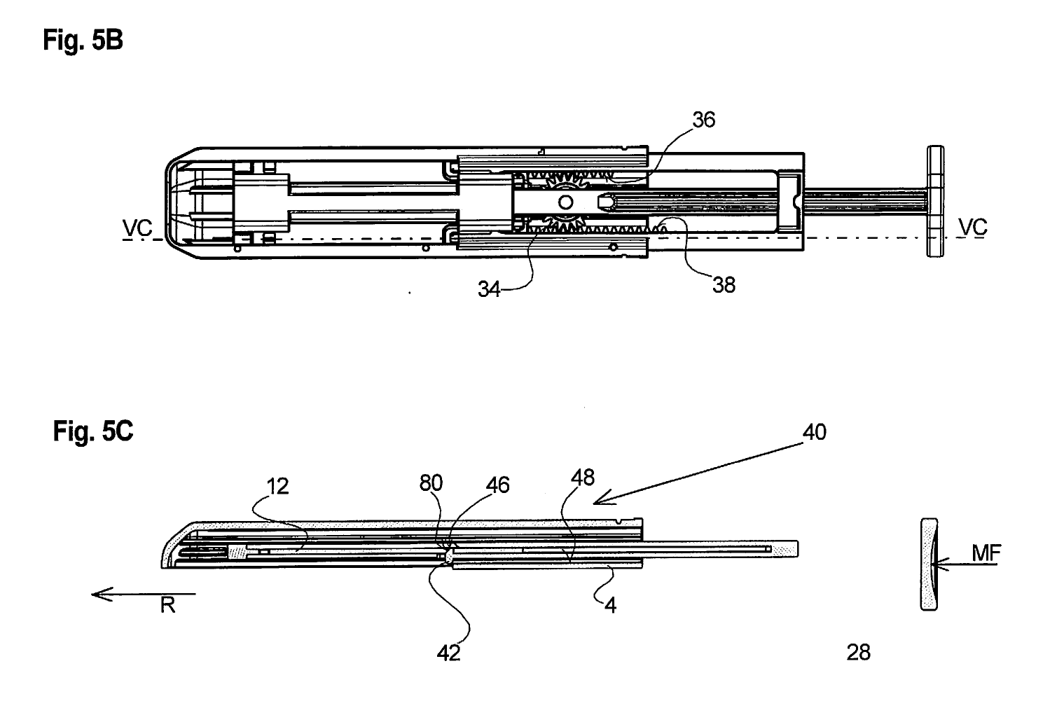

FIG. 5A a perspective view of the injection device according to FIG. 4A, with the cover removed, after carrying out an insertion stroke,

FIG. 5B a plan view of the injection device according to FIG. 5A,

FIG. 5C a cut-away view of the injection device in the plane VC-VC of FIG. 5B,

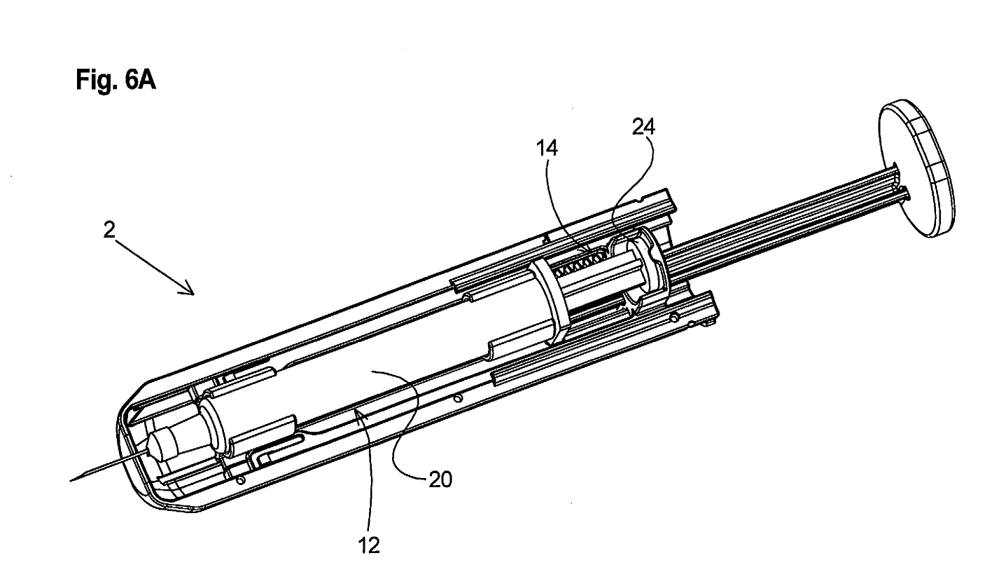

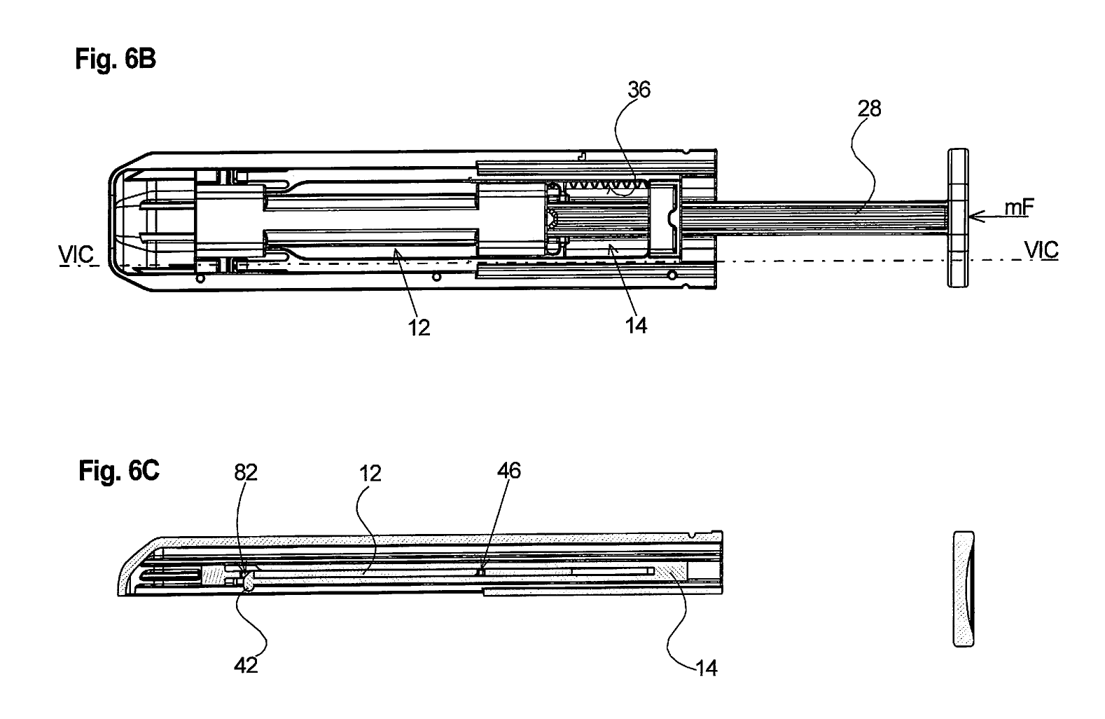

FIG. 6A a perspective view of the injection device according to FIG. 4A, with the cover removed, after carrying out an injection stroke,

FIG. 6B a plan view of the injection device according to FIG. 6A,

FIG. 6C a cut-away view of the injection device in the plane VIC-VIC of FIG. 6B,

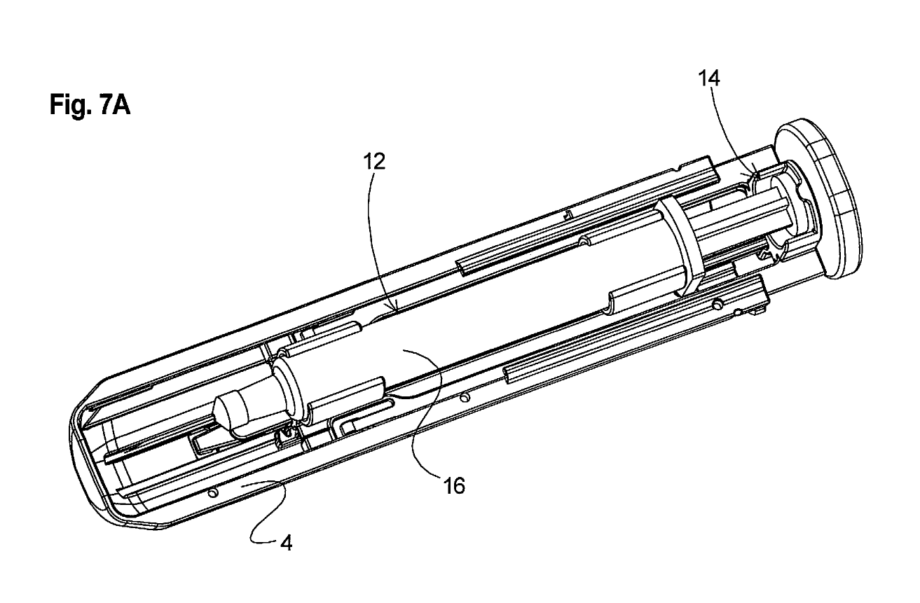

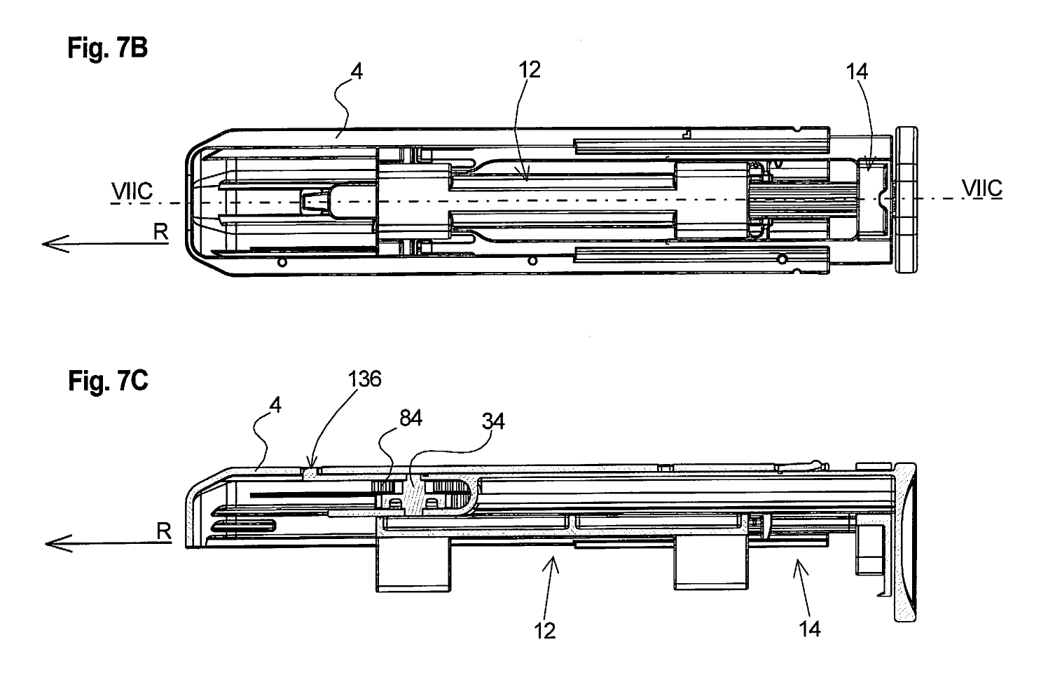

FIG. 7A a perspective view of the injection device according to FIG. 4A, with the cover removed, after carrying out a return stroke,

FIG. 7B a plan view of the injection device according to FIG. 7A,

FIG. 7C a cut-away view of the injection device in the plane VIIC-VIIC of FIG. 7B,

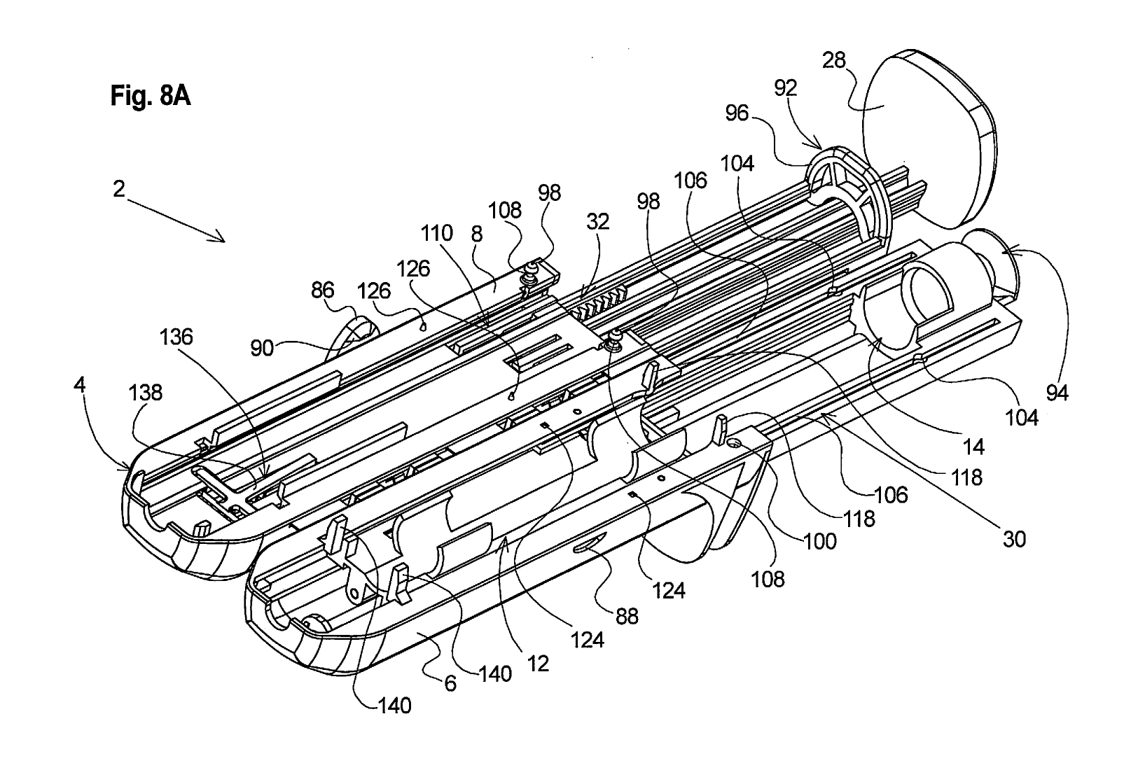

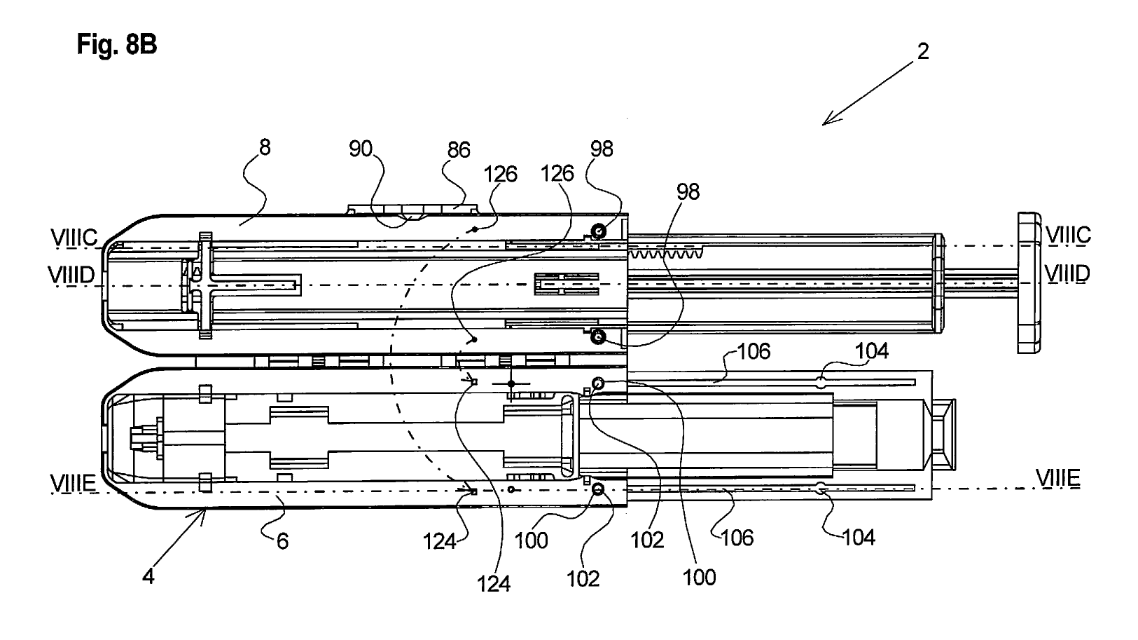

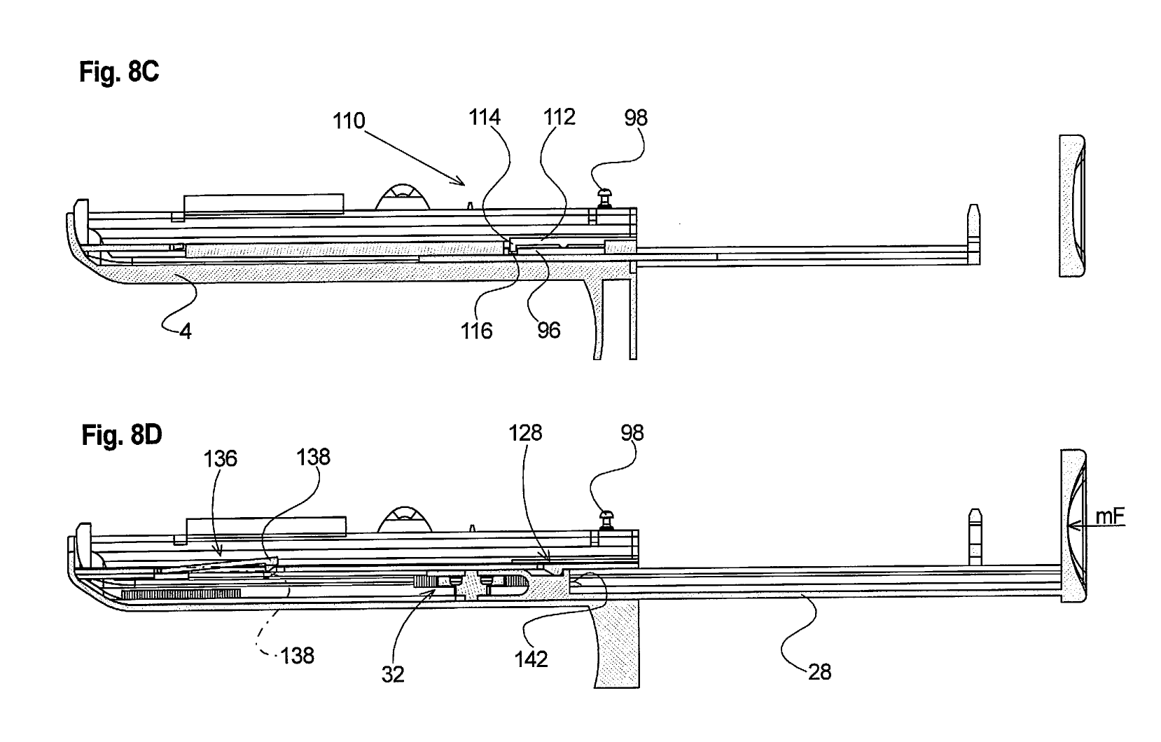

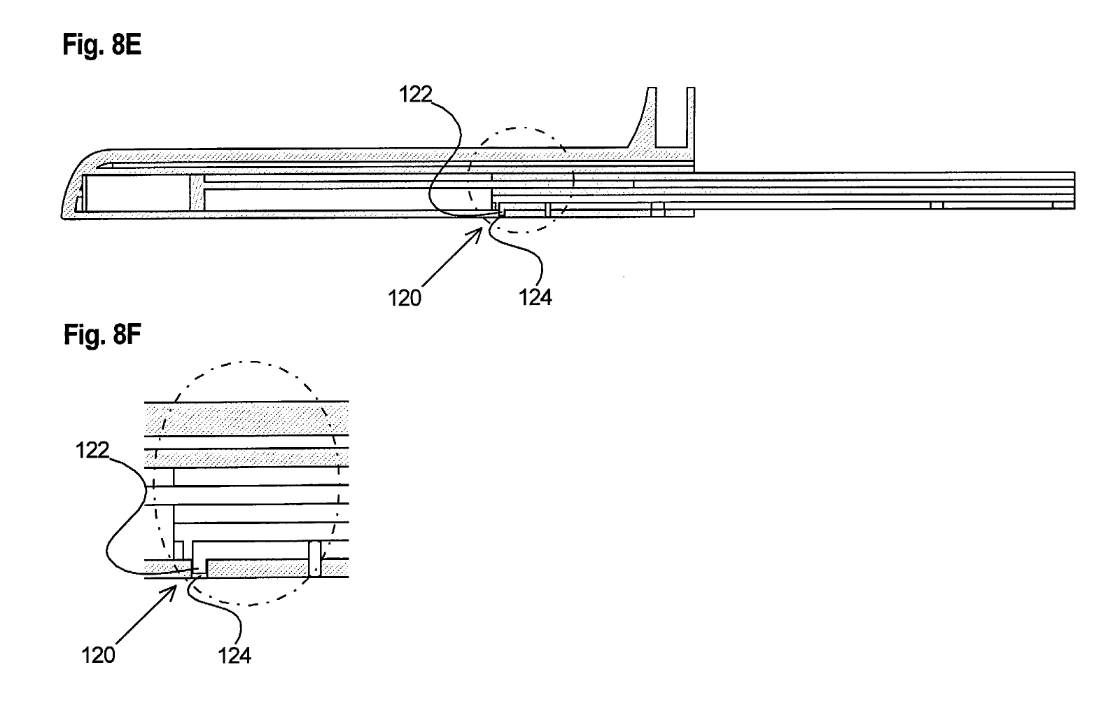

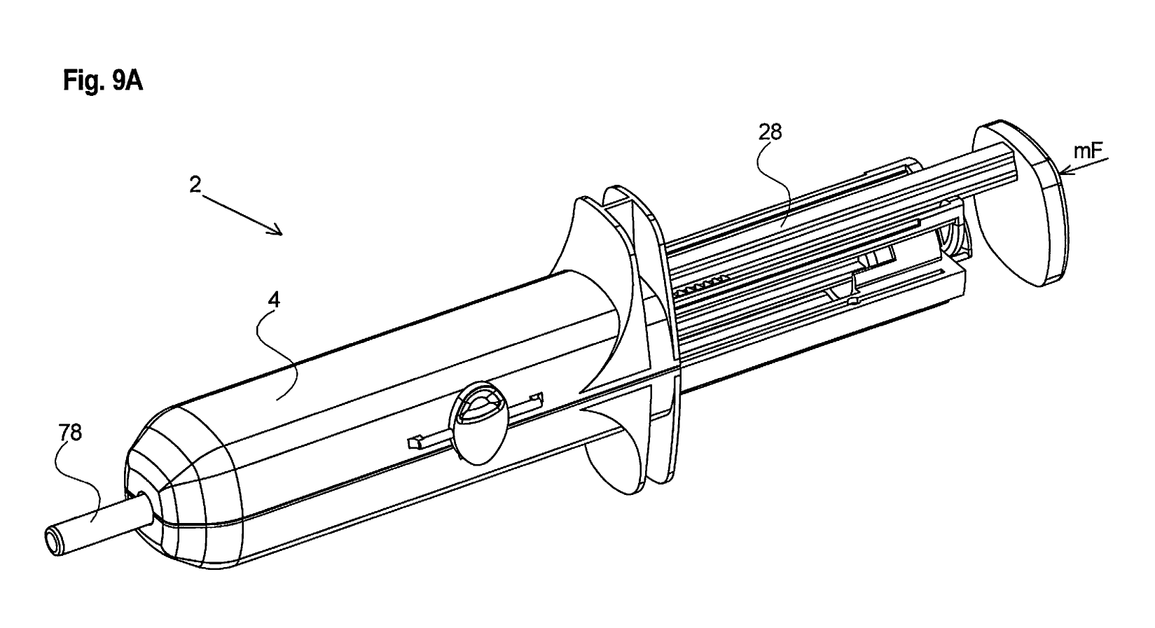

FIG. 8A a perspective view of an alternative embodiment of the injection device, in the open position,

FIG. 8B a plan view of the injection device according to FIG. 8A,

FIG. 8C a cut-away view of the injection device in the plane VIIIC-VIIIC of FIG. 8B,

FIG. 8D a cut-away view of the injection device in the plane VIIID-VIIID of FIG. 8B,

FIG. 8E a cut-away view of the injection device in the plane VIIIE-VIIIE of FIG. 8B,

FIG. 8F an enlarged view of fixing means of the injection device from FIG. 8E,

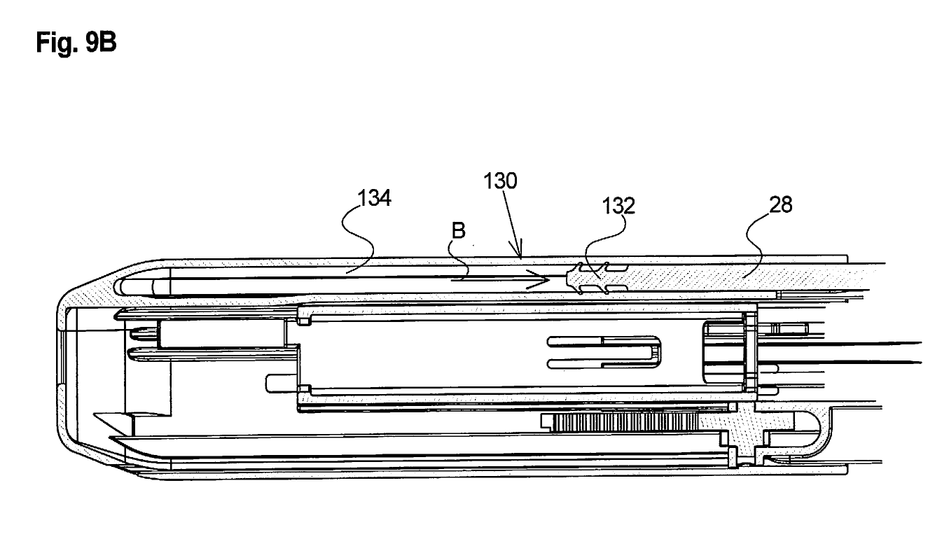

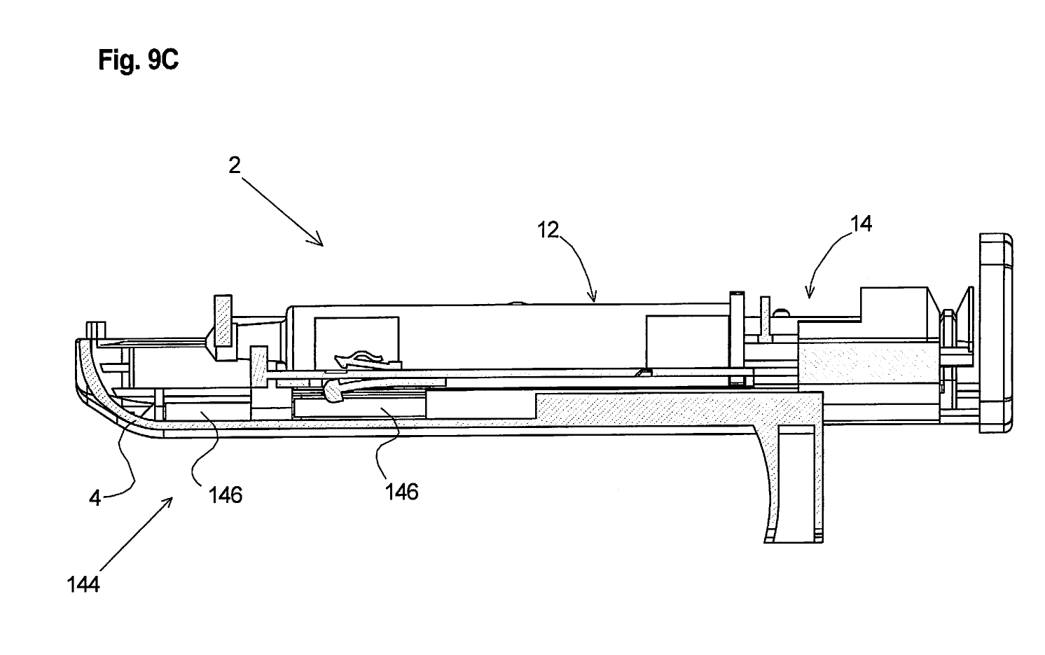

FIG. 9A a perspective view of the injection device according to FIG. 8A in the closed position,

FIG. 9B a cut-away view of a damping device of the injection device from FIG. 9A and

FIG. 9C a cut-away view of a displacement mechanism of the injection device from FIG. 9A.

FIG. 1 shows an injection device 2 with a housing 4 that has a first outer housing part 6 and a second outer housing part 8. The two outer housing parts 6, 8 are connected to each other by a pivot bearing 10, by means of which they can be displaced between an open position and a closed position.

In the illustrated open position, a syringe holder 12 and a plunger holder 14 of the injection device 2 are freely accessible. As can be seen from FIG. 2A in particular, this permits a syringe 16 to be accommodated, that is filled with a medicament M to be administered and has an injection needle 18, a barrel 20, a piston 22 and a syringe plunger 24. The barrel 20 is held in the syringe holder 12 and the syringe plunger 24 in the plunger holder 14. The barrel 14 forms, through lateral support means 25, a contact portion, in which the free end of the syringe plunger 24 is housed in a self-centring manner.

To carry out a controlled application procedure, in which the syringe 16 is pressed and the medicament M is administered to a patient, the injection device 2 has a control assembly that is generally designated 26. This enables the carrying out of an injection stroke and a return stroke simply by applying a manual force mF to an actuation element 28, in an injection direction R.

For this purpose, the control assembly 26 has a transmission device 30 with a gear drive 32, which couples the actuation element 28 to the syringe holder 14, as can be seen from FIG. 2B and 2C. As a result, the plunger housing 14 can be driven, in accordance with movement of the actuation element 28 relative to the housing 4. To achieve this, the gear drive 32 is provided with a gear wheel 34 that is rotatably mounted on the actuation element 28 and that can intermesh with at least one toothed rack 36 on the housing side as well as with a toothed rack 38 on the plunger housing side.

Moreover, the control assembly 26 has a connecting mechanism 40 that can be seen in FIG. 2D. Here, a detent element in the form of a latch hook 42 is provided, which is attached to the plunger housing 14 by means of an elastic arm 44. In the illustrated starting position, this latch hook 42 is in relaxed engagement with a first detent housing 46 of the syringe holder 12 and is thus on the inside 48 of the housing 4, which acts as the control profile.

As can be seen in particular from the enlarged representation of the pivot bearing 10 in FIG. 3A, biasing means 50 are provided between the first outer housing part 6 and the second outer housing part 8, by means of which the two outer housing parts 6, 8 can be biased, in both the illustrated open position and in the closed position. The biasing means 50 are formed, for example, by springy connecting elements in the form of butterfly hinges. Alternatively or in addition, simple film hinges 52 can also be provided between the first outer housing part 6 and the second outer housing part 8, as shown by way of example in FIG. 3B. In every such case, both outer housing parts 6, 8 must be firmly fixed to each other or must be formed as one piece. It is also possible to construct the pivot bearing 10 in such a way that it acts as a defined breaking point that breaks after a predetermined number of opening and closing procedures to indicate that the overall injection device 2 has worn out.

Alternatively, the pivot bearing 10 can also be formed by hinges 64a, 64b that engage with each other, as shown in FIG. 3C and 3D. Thus, it is possible to manufacture the two outer housing parts 6, 8 separately from each other and then to engage them with each other to form the housing 4.

In any case, as can further be seen from FIG. 3A, additional stop means 54 can be provided, to form an end-stop on the pivot bearing 10, and to prevent the pivot bearing 10 from being overloaded, in particular when pivoting to the open position.

Furthermore, several guide slots 60 are set into the first outer housing part 6. Guide pins 58 are provided in corresponding positions on the second outer housing part 8. As can be seen from FIG. 3E and 3F, these are inserted in guide slots 60 when the housing 4 is closed. In the closed position of the housing 4, these guide slots 60 together with the guide pins 58 they hold also act as a means of relieving strain transverse to the pivot direction.

In every case, it is envisaged that in the embodiment of the injection device 2 according to FIGS. 1 to 3 the two outer housing parts 6, 8 can be permanently locked together in the closed position. The permanent connection between the two outer housing parts 6, 8 thus achieved can ensure use of the injection device 2 purely as a single-use product.

For this purpose, for example, a finger support 66 can be provided, which is slid onto the housing 4 in the closed position, thus surrounding the two outer housing parts 6, 8 in a ring, as shown in FIG. 4A. To ensure a permanent fixed connection of the two outer housing parts 6, 8, a permanent snap connection 68 is, for instance, provided between the finger support 66 and the housing 4. As shown in FIG. 4B, this can for example have detent elements 68a on the support side that can be engaged on detent holders 68b on the housing side, or vice versa. Alternatively, permanent connecting means can be provided directly between the two outer housing parts 6, 8 (not shown).

As can also be seen from FIGS. 4C and 4D, the injection device 2 has an actuation lock 70, by means of which movement of the actuation element 28 is blocked in the open position of the housing 4. For this purpose, the actuation lock 70 has a pivotable lever 72 on which a lug 74 is formed and which is biased to an engagement position with an engagement housing 76 set into the actuation element 28.

By sliding the finger support 66 onto the housing 4, the lever 72 is pivoted so that the lug 74 disengages with the engagement housing 76 and the injection device 2 is thus ready for use. After removing a protective cap 78 from the injection needle 18 and placing the front end of the injection device 2 onto a patient, an injection procedure can thus be initiated by applying manual force mF to the actuation element 28—see FIG. 4A—for instance by the patient's thumb (not shown).

In order to be able to set a predefined threshold limit for the force mF, at the attainment of which the injection procedure is started, a start actuator 128 in the form of an elastic, pivotable latch hook can be provided, as shown in FIG. 4C, that disengages from the housing 4 when this threshold limit is reached and thus only then enables movement of the actuation element 28.

There follows the insertion stroke, in which the gear wheel 34 engages both with the toothed rack 36 on the housing side and with the toothed rack 38 on the plunger holder side, with the result that the injection device 2 is moved to the position shown in FIGS. 5A to 5C. In this position, the injection needle 18 projects from the housing 4. At the same time, the latch hook 42 of the connecting mechanism 40 has now moved so far that it is no longer in contact with the inner side 48 of the housing 4. By the further application of manual force mF to the actuation element 28, due to an oblique surface 80 formed on the front of it, that presses against the syringe holder 12, the latch hook can now be laterally pivoted from the detent housing 46 and moved relative to the syringe holder 12 further in the injection direction R.

This is now followed by the injection stroke, in which the plunger holder 14, with the syringe plunger 24 received in it, is moved relative to the syringe holder 12 with the barrel 20 received in it, to press out the medicament M, until the position of the injection device 2 shown in FIGS. 6A to 6C is reached. In this position, the gear wheel 34 disengages from the toothed rack 36 on the housing side so that the further movement of the actuation element 28 is not transmitted to the syringe holder 12 and/or the plunger holder 14 and an idle stroke is thus executed. In addition, the latch hook 42 of the connecting mechanism 40 is positioned at the end of the injection stroke at the level of a second detent housing 82 of the syringe holder 12, with which it engages due to its elastic return forces.

As a result of the continued movement of the actuation element 28 in injection direction R, after completing the idle stroke the gear wheel 34 engages with a second toothed rack 84 on the housing side that is arranged on one of the sides opposite the first toothed rack 36 on the housing side (see FIG. 2C). As a consequence, the toothed rack 34, which simultaneously engages with the toothed rack 38 on the plunger holder side, is turned in the opposite direction from previously and thus moves both the plunger holder 14 and the syringe holder 12, the movement of which is coupled to the plunger holder 14 by the latch hook 42 and the detent housing 82, against the injection direction R. This recovery stroke is completed according to FIGS. 7A to 7C, until the syringe 16 that has now been pressed is once again completely located within the housing 4. In this end position, the start actuator 128 in the form of a latch hook can engage with a locking recess 142 according to FIG. 4C and is thus once again fixed relative to the housing in order to prevent fresh activation of the injection device via the actuation element.

FIGS. 8A and 8B show an alternative embodiment of the injection device 2, which enables replacement of the syringe 16 housed in the housing 4 and thus multiple use of the injection device 2. For this, a detachable closing assembly with a closure element 86 is provided that can be releasably engaged with a closure holder 88 to connect the first outer housing part 6 to the second outer housing part 8 in the closed position. In relation to this, the closure element 86 has a flexibly mounted snap hook 90 that together with the closure holder 88 forms a releasable snap connection. Alternatively, any other known and suitable releasable connection between the two outer housing parts 6, 8 could be used, such as a frictional connection. In addition, releasable connection of the outer housing parts 6, 8 in the closed position could be contemplated using the sliding finger support 66, which in this case interacts with the housing 4 in such a way that it can be removed from the housing 4 after an injection procedure has been completed (not shown).

Also, as can be seen from 8A and 8B, the syringe holder 12 and the plunger holder 14 in this embodiment of the injection device 2 are stored on the first outer housing part 6, whereas the actuation element 28 is retained on the second outer housing part 8 together with the gear drive 32. Here, the transmission device 30 forms, between the plunger holder 14 and the actuation element 28, a coupling device 92 that is disconnected in the illustrated open position of housing 4.

The coupling device 92 has a coupling means 94 on the plunger holder side in the form of a groove and a counter-coupling means 96 on the actuation element side that is formed by a tappet held on the actuation element 28. In the illustrated open position of the housing 4, the coupling means 94 on the plunger holder side is spaced apart from the counter-coupling means 96 on the actuation element side.

By pivoting the two outer housing parts 6, 8 to the closed position, the coupling means 94 can positively engage with the counter-coupling means 96, provided that both elements are correspondingly arranged at the same level. The counter-coupling means 96 is thereby partially formed around the actuation element 28 so that in the closed position it is supported on the plunger holder 14 by the counter-coupling means 96.

To ensure that the housing 4 can be moved to the closed position only in the precise, illustrated starting position of the plunger holder 14, securing pins 98 are provided on the second outer housing part 8. These can each cooperate with an opening 100 on the first outer housing part 6 with a first entry opening 102 that together with a second entry opening 104 is inset along a securing groove 106 in the plunger holder 14.

The securing pins 98 act as encoding means that can only be inserted into the first entry opening 102 with exact arrangement of the first entry openings 102 at the level of the openings 100, in order to close the housing 4.

Moreover, the securing pins 98 each have an undercut 108 that is dimensioned so that the edges of the securing pins 98 engage in the corresponding securing groove 106 and can be moved relative to the groove towards the second entry opening 104. As a result, the securing pins 98 together with the securing grooves 106 also form a safety mechanism which can prevent the housing 4 being moved into the open position while the actuation element 28 is being moved.

Together with the second entry openings 104, the securing pins 98 in turn act as encoding means that only permit the housing 4 to be moved into the open position after an application procedure has been carried out if the entry openings 104 are at exactly the level of the openings 100.

To prevent movement of the actuation element 28 in the open position of the housing 4, there are also provided fixing means 110, by means of which the counter-coupling device 96 and consequently also the actuation element 28 are fixed relative to the housing 4 in the starting position, as long as it is in the illustrated open position.

As can be seen from FIG. 8C in particular, these fixing means 110 each have a rocker 112 on the housing side. In the open position of the housing 4, a blocking hook 114 formed on a first end of the rocker engages with a hook holder 116 that is inset in the counter-coupling means 96 that functions as a tappet. When the housing 4 is moved to the closed position, the rockers 112 are actuated by a release tab 118 on a second end that protrudes from the other outer housing part 6, 8, as can be seen from FIG. 8A in particular. This actuation pivots the rocker 112 so that the blocking hook 114 disengages from the corresponding hook holder 116. In this way, blocking of the actuation element 28 by the counter-coupling means 96 in the closed position is cancelled.

Moreover, to prevent movement of the syringe holder 12 and the plunger holder 14 in the open position of the housing 4, there are provided two fixing means 120 with a second blocking hook 122, by means of which the plunger holder 14 engages in the open position with a housing opening 124. As a result, the plunger holder 14 and, via the connecting mechanism 40 also the syringe holder 12, are fixed in the open position relative to the housing 4 in its starting position.

The engagement of the plunger holder 14 on one of the outer housing parts 6, 8 can be cancelled when the housing 4 is moved into the closed position by a protruding unlocking pin 126 on the other outer housing part 8, 6 by means of which the second blocking hook 122 can be pressed out of the housing opening 124 (see FIG. 8F).

After accepting the syringe 16 and movement of the housing 4 to the closed position described above, the injection device 2 is in ready-for-use condition of FIG. 9A. After removal of the protective cap 78 and placement of the front end of the injection device 2 on the patient concerned, the actuation element 28 can now have the manual force mF applied to it, to carry out the injection procedure in line with the method described above in relation to the embodiment of FIGS. 1-7.

As can be seen from FIG. 8D, at the start of the injection procedure, the manual force mF must be great enough to disengage the actuation element 28 from the start actuator 128, which is formed by a snap connection between the housing 4 and the actuation element 28. As soon as the resistance defined by the start actuator 128 is overcome by the manual force mF, the injection procedure than follows the sequence described above.

As can be seen from FIG. 9B, the injection device 2 has a damping device 130 to keep the propulsion speed of the actuation element 28 in a predefined range while the manual force mF is being applied. For this, the damping device 130 has a displacer 132, the movement of which is coupled to the actuation element 28. During displacement of the actuation element 28, the displacer is moved along an airspace 134, whereby a body of air contained within it has to be driven out. A predefined braking force B is generated by this air displacement that is opposed to the manual force mF.

After completion of the injection procedure, the actuation element 28 is fixed by an end-position lock 136 at the end of the return stroke, whereby drawing-up of the injection device 2 in the closed position is prevented. As can be seen from FIG. 8A in particular, the end position lock 136 has a hook arm 138 which is moved by two clamping spigots 140 from a release position to an active position when the housing 4 is closed (see FIG. 8D). In this active position, the hook arm 138 engages with a locking recess 142 of the actuation element 28 as soon as the latter occupies its end position after completion of the return stroke.

To release the end-position lock 136, the housing 4 must first of all be brought into the open position, whereby the hook arm 138 moves back to the release position due to elastic return forces. Then, the actuation element 28 can once again be moved relative to the housing 4 to its starting position, so that a new injection procedure can be carried out after the syringe 16 has been replaced.

As can be seen from FIG. 9C, the syringe holder 12 and the plunger holder 14 can also be biased in the starting position by a displacement mechanism 144. In the illustrated embodiment of the injection device 2, this displacement mechanism 144 is formed by two spring devices 146 that act between the housing 4 and the syringe holder 12 or the plunger holder 14. By virtue of the coupling between the syringe holder 12 and the plunger holder 14 that is cancelled when the housing 4 is opened, and the simultaneous disconnection of the coupling device 92, the spring devices 146 can automatically press the syringe holder 12 and the plunger holder 14 into their respective starting positions.

In this starting position that has now been adopted, a new syringe 16 can be inserted, to enable the next injection procedure to be carried out.

So that syringes 16 of different lengths can be used, it may be possible to use adapter pieces (not shown) that are inserted in the syringe holder 12 and/or in the plunger holder 14. Alternatively, it is also possible to make the syringe holder 12 and/or plunger holder 14 exchangeable so that different sizes of these components can be utilised in the injection device 2 (not shown).

These measures—illustrated for the multiple use injection device 2—for adapting to different syringe sizes, as well as the safety measures described above for the avoidance of mis-use, can also be used in relation to the injection device 2 configured as a single-use product according to FIGS. 1 to 7.

1-40. (canceled)

41. An injection device, comprising:
a housing in which a syringe can be held, the syringe having an injection needle, a barrel, a piston and a syringe plunger;
a control assembly for the controlled actuation of the syringe during an application procedure comprising at least an insertion stroke, an injection stroke and a return stroke, wherein the control assembly has an actuation element to be applied by a manual driving force;
a syringe holder which can be displaced in relation to the housing, and to which the barrel can be fastened;
a plunger holder which can be displaced in relation to the housing, and to which the syringe plunger can be fastened; and
a transmission device with a gear drive to couple the plunger holder to the actuation element, by means of which gearing the plunger holder can be driven, in accordance with a movement of the actuation element relative to the housing,
wherein the housing has a first outer housing part and a second outer housing part, which are retained on each other by means of a pivot bearing provided therebetween and are displaceable between an open position in which the syringe holder is accessible and a closed position in which the syringe holder is enclosed,
wherein the transmission device is provided with a coupling device between the plunger holder and the actuation element with a coupler on the plunger holder side and counter-coupler on the actuation element side, which is disconnected in the open position.

42. The injection device according to claim 41, wherein the two outer housing parts can be biased against each other both in the open position and in the closed position.

43. The injection device according to claim 41, wherein the second outer housing part can be connected permanently or releasably to the first outer housing part in the closed position.

44. The injection device according to claim 41, wherein an end-stop is formed between the first outer housing part and the second outer housing part, by means of which the two housing parts can be positioned next to each other in the open position.

45. The injection device according to claim 41, wherein the first outer housing part and the second outer housing part are guided next to each other when pivoting from the open position to the closed position.

46. The injection device according to claim 41, wherein the pivot bearing forms a defined breaking point.

47. The injection device according to claim 41, wherein an actuation lock is provided, which blocks movement of the actuation element in the open position.

48. The injection device according to claim 41, wherein the syringe holder and the plunger holder are held on the first outer housing part and the actuation element and the gear drive are held on the second outer housing part.

49. The injection device according to claim 41, wherein the coupler on the plunger holder side and counter-coupler on the actuation element side, can in the closed position be positively engaged in and counter to the injection direction.

50. The injection device according to claim 41, wherein an adapter is provided, by which the syringe holder and/or the plunger holder is adaptable to different syringes.

51. The injection device according to claim 41, wherein and encoder is provided between the outer housing parts and the syringe holder and/or the plunger holder, which defines a starting position in which the housing parts can be moved to the closed position.

52. The injection device according to claim 41, wherein a safety mechanism is provided between the first outer housing part and the second outer housing part to prevent the two housing parts from opening during operation of the injection device.

53. The injection device according to claim 41, wherein a releasable connecting mechanism is arranged between the syringe holder and the plunger holder, with a detent element that can be engaged with a detent holder in an insertion position and in a return position.

54. The injection device according to claim 51, wherein a displacement mechanism is provided, by means of which the plunger holder can be automatically brought into the starting position by movement of the outer housing parts to the open position.

55. The injection device according to claim 41, wherein the actuation element is coupled to a damping device.

56. The injection device according to claim 41, wherein an end-position lock is provided, to hold the actuation element in an end-position taken up at the end of an injection procedure, and the end-position lock can be brought to a release position by moving the outer housing parts into the open position.

57. The injection device according to claim 42, wherein the second outer housing part can be connected permanently or releasably to the first outer housing part in the closed position.

58. The injection device according to claim 42, wherein an end-stop is formed between the first outer housing part and the second outer housing part, by means of which the two housing parts can be positioned next to each other in the open position.

59. The injection device according to claim 43, wherein an end-stop is formed between the first outer housing part and the second outer housing part, by means of which the two housing parts can be positioned next to each other in the open position.

60. The injection device according to claim 42, wherein the first outer housing part and the second outer housing part are guided next to each other when pivoting from the open position to the closed position.
